(19) United States
(12) Patent Application Publication
(10) Pub. No.: US 2025/0261987 A1
(43) Pub. Date: Aug. 21, 2025

(54) Surgical Instruments including Expandable Electrodes and Methods for Ablating Tissue in a Patient with the Surgical Instruments

(71) Applicant: PainLab Ltd., London (GB)

(72) Inventors: Thomas L. Yearwood, London (GB); Stephen Patrick Ward, London (GB)

(21) Appl. No.: 18/443,944

(22) Filed: Feb. 16, 2024

(51) Int. Cl. A61B 18/14; A61B 17/00; A61B 17/34; A61B 18/00

(52) U.S. Cl. CPC A61B 18/148; A61B 2017/00867; A61B 17/3415; A61B 2018/00077; A61B 2018/00083; A61B 2018/00267; A61B 2018/00577

(57) ABSTRACT

An example surgical instrument includes an insulated shaft, and an expandable electrode at a distal end of the insulated shaft. The expandable electrode includes a shape memory alloy lattice. Based on passing electrical current through the shape memory alloy lattice, the shape memory alloy lattice undergoes a shape memory transition from a non-expanded configuration to an expanded configuration causing the expandable electrode to radially expand outward in diameter.

### FIELD

The present disclosure is generally related to a surgical instrument including an insulated shaft, and an expandable electrode at a distal end of the insulated shaft activated by passing electrical current through the electrode to cause a shape memory transition of the expandable electrode from a non-expanded configuration to an expanded configuration. The present disclosure also relates to methods for ablating tissue in a patient with the surgical instrument.

### BACKGROUND

Radiofrequency (RF) procedures in pain management use alternating current in the AM RF band to interrupt or modulate neural pain pathways, usually between 50-500 kHz. RF current is a high-frequency (50-500 kHz) alternating current that causes adjacent tissue molecules to oscillate and generate heat. If applied to adjacent tissue for a sufficient duration, enough heat is generated to coagulate the tissue. For example, continuous applied RF generates thermal coagulation of neural tissue to interrupt pain pathways, and pulsed RF modulates neural transmission without nerve injury.

Both continuous and pulsed RF have been used to treat spinal pain (e.g., facet RF, disc RF, sacroiliac RF) and neuropathic pain syndromes, including trigeminal neuralgia, occipital neuralgia, and a range of peripheral neuropathies and nerve entrapment conditions.

Conventionally, RF procedures includes placing a needle electrode alongside a target nerve (usually under radiographic screening). The needle has an uninsulated tip (usually 2-15 mm in length), and heat energy is produced along this active tip. A sufficient lesion (coagulation) diameter is highly dependent on a size of the electrode. For example, increasing a cannula diameter from 22 to 16 gauge increases average lesion width by 58-65% (3-4 mm) at 80 degrees C. and 2 minutes.

For many RF procedures, larger lesion sizes are critical to success. Still, a requirement for a large lesion created by a large cannula must be balanced against a risk of needle injury and tissue pain inherent in a process of inserting such large cannulae into a patient.

### SUMMARY

Within examples, a surgical instrument is described, comprising an insulated shaft, and an expandable electrode at a distal end of the insulated shaft. The expandable electrode comprises a shape memory alloy lattice, and based on passing electrical current through the shape memory alloy lattice, the shape memory alloy lattice undergoes a shape memory transition from a non-expanded configuration to an expanded configuration causing the expandable electrode to radially expand outward in diameter.

Within further examples, a surgical equipment is described comprising an introducer, and a surgical instrument inserted into the introducer. The surgical instrument comprises an insulated shaft, and an expandable electrode at a distal end of the insulated shaft. The expandable electrode comprises a shape memory alloy lattice, and based on passing electrical current through the shape memory alloy lattice, the shape memory alloy lattice undergoes a shape memory transition from a non-expanded configuration to an expanded configuration causing the expandable electrode to radially expand outward in diameter.

Within further examples, a method of ablating tissue in a patient with a surgical instrument is described. The method comprises inserting a trocar needle through an introducer and into the patient allowing the introducer to be positioned adjacent to a target area, removing the trocar needle from the introducer, and inserting the surgical instrument into the introducer. The surgical instrument comprises an insulated shaft and an expandable electrode at a distal end of the insulated shaft, and the expandable electrode comprises a shape memory alloy lattice. The method also comprises exposing the expandable electrode relative to the introducer causing the expandable electrode to protrude out from the introducer, applying electrical current to pass through the shape memory alloy lattice of the expandable electrode to cause the shape memory alloy lattice to undergo a shape memory transition from a non-expanded configuration to an expanded configuration so that the expandable electrode radially expands outward in diameter, and contacting the tissue with at least a portion of the expandable electrode exposed from the introducer while in the expanded configuration.

Example implementations described herein would, for example, allow the passage of a 22 gauge needle/introducer and, following an expansion of the electrode tip, an RF lesion equivalent to that created by a 16 gauge needle is provided. This provides greater patient tolerability during needle insertion, a lower risk of unintended damage to local tissue, and improved therapeutic results.

These as well as other aspects, advantages, and alternatives will become apparent to those of ordinary skill in the art by reading the following detailed description, with reference where appropriate to the accompanying drawings.

### BRIEF DESCRIPTION OF THE DRAWINGS

Many aspects of the disclosure can be understood with reference to the following drawings. The components in the drawings are not necessarily to scale. Moreover, in the drawings, like reference numerals designate corresponding parts throughout the several views.

FIG. 1 illustrates an example of a surgical instrument including an expandable electrode in a compressed form, according to an example implementation.

FIG. 2 illustrates an example of the surgical instrument of FIG. 1 including the insulated shaft without the insulation and the expandable electrode formed on the same conductor as a single integral component, according to an example implementation.

FIG. 3 illustrates an example of a shape memory alloy lattice of the expandable electrode, according to an example implementation.

FIG. 4 illustrates the shape memory alloy lattice comprising a plurality of geometric unit cells repeated in circumferential and axial directions along a longitudinal axis of the expandable electrode, according to an example implementation.

FIG. 5 illustrates an example of the expandable electrode in an expanded configuration, according to an example implementation.

FIG. 6 illustrates another example of the expandable electrode in an expanded configuration, according to an example implementation.

FIG. 7 illustrates another example of the expandable electrode shown in FIG. 6, according to an example implementation.

FIG. 8 illustrates an example of surgical equipment, according to an example implementation.

FIG. 9 illustrates the surgical equipment of FIG. 8 with the expandable electrode in the expanded configuration.

FIG. 10 illustrates comparative dimensions of the expandable electrode in the non-expanded configuration and the expanded configuration, according to an example implementation.

FIG. 11 illustrates another example of the surgical equipment, according to an example implementation.

FIG. 12 illustrates an example of the surgical equipment with the expandable electrode in an extended and retracted position, according to an example implementation.

FIG. 13 shows a flowchart of an example of a method of ablating tissue in a patient with a surgical instrument, according to an example embodiment.

FIG. 14 illustrates an example of inserting a trocar needle through the introducer, which is then subsequently inserted into a patient allowing the introducer to be positioned adjacent to a target area, according to an example implementation.

FIG. 15 illustrates an example of inserting the surgical instrument into the introducer, according to an example implementation.

FIG. 16 illustrates another example of inserting the expandable electrode into the introducer, according to an example implementation.

FIG. 17 illustrates yet another view of inserting the surgical instrument into the introducer, according to an example implementation.

FIG. 18 illustrates an example of stages of exposing the expandable electrode relative to the introducer, according to an example implementation.

FIG. 19 illustrates yet another view of stages of exposing the expandable electrode relative to the introducer, according to an example implementation.

FIG. 20 illustrates application of the surgical equipment for treatment of pain for RF ablation of a lumbar facet joint with the surgical instrument inserted into the introducer and the expandable electrode is exposed and in the non-expanded configuration, according to an example implementation.

FIG. 21 illustrates a magnified view of FIG. 20 of the exposed metallic contact of the expandable electrode that will allow passage of RF current to the nerve, according to an example implementation.

FIG. 22 illustrates application of the surgical equipment for treatment of pain for RF ablation of a lumbar facet joint with the surgical instrument inserted into the introducer and the expandable electrode is exposed and in the expanded configuration, according to an example implementation.

FIG. 23 illustrates a magnified view of FIG. 22 of the expandable electrode in expanded form and lying parallel to the medial branch nerve, according to an example implementation.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Example methods, devices, and systems described herein provide an expandable radiofrequency (RF) electrode for the treatment of pain in subjects/patients. RF current is a high-frequency (50-500 kHz) alternating current, which when applied to a target area causes adjacent tissue molecules to oscillate and generate heat. If the RF current is applied to adjacent tissue for a sufficient duration, enough heat is generated to coagulate the tissue. Conventionally, a needle electrode is placed alongside a target nerve (usually under radiographic screening), and the needle has an uninsulated tip (e.g., 2-15 mm in length) enabling heat energy to be produced along this active tip and not distal to it. A surface temperature of the RF needle is monitored with a thermistor to avoid tissue damage by heating tissue to the temperatures of cavitation (>100° C.).

For many RF procedures, larger lesion sizes are critical to success, but a requirement for a large lesion created by a large cannula must be balanced against the risk of needle injury and tissue pain inherent in the process of inserting these large cannulae. Within examples herein, an expandable RF electrode addresses this issue. For example, surgical equipment including an insulated introducer, a trocar needle, and a surgical instrument with the expandable RF electrode is described.

In an example application, the trocar needle is inserted through the introducer allowing the introducer to be positioned adjacent to the target area. Once in position, the trocar needle is removed, and the surgical instrument including the expandable RF electrode is passed through the introducer. As the intruder is retracted (e.g., by about 10-15 mm, where “about” includes distances or measurements of more or fewer than 2-3 mm), a distal end of the expandable RF electrode emerges at a distal extent of the introducer.

The expandable RF electrode comprises an insulated shaft and, at an end of the tip, an elongated shape memory alloy lattice. The shape memory alloy lattice structure is compressible so that it may pass through a lumen of the introducer. When the lattice portion emerges from the distal end of the introducer, the super-elastic shape memory properties of the shape memory alloy enable the lattice structure to expand to a pre-formed elongated shape based on application of an electrical current.

Following, RF current is applied along the electrode employing an RF generator system. The lattice portion of the electrode receives the RF current and generates heat to adjacent tissue-lattice arrangements behave in the same way as solid surfaces in terms of electromotive field emission, and a lesion is formed circumferentially only along the body of the lattice and not distal to it.

In some examples, the surgical equipment allows a passage of a 22 gauge needle/introducer, and following an expansion of the electrode tip, an RF lesion equivalent to that created by a 16 gauge needle is provided. This provides greater patient tolerability during needle insertion, a lower risk of unintended damage to local tissue, and improved therapeutic results.

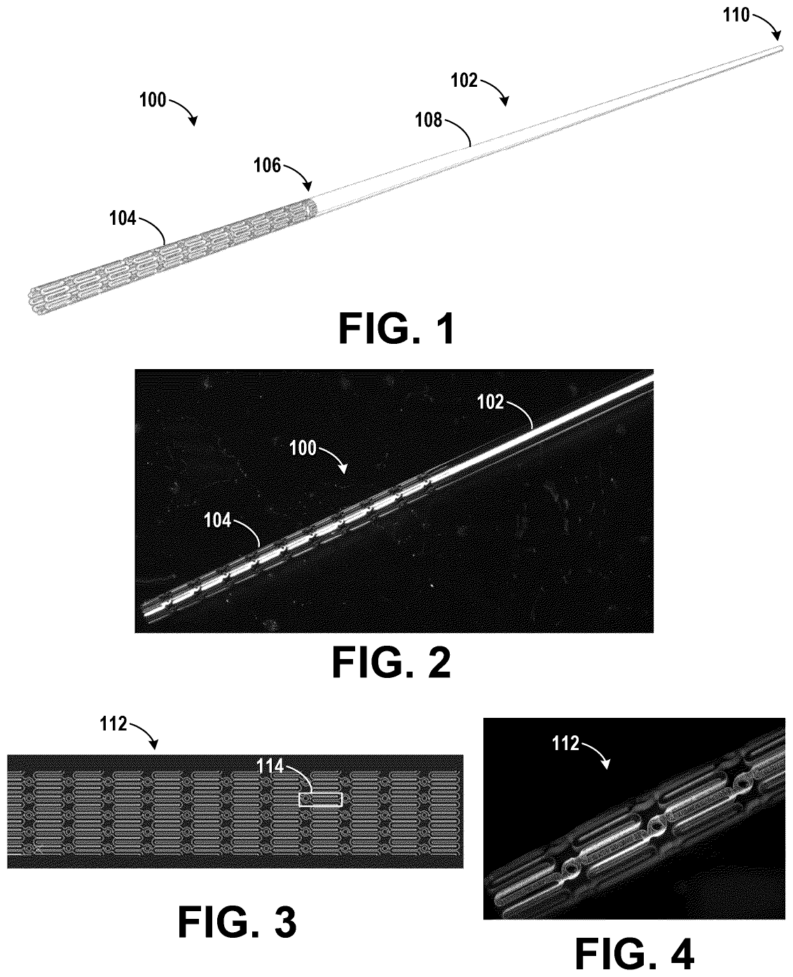

Referring now to the figures, FIG. 1 illustrates an example of a surgical instrument 100 including an expandable electrode in a compressed form, according to an example implementation. The surgical instrument 100 comprises an insulated shaft 102, and an expandable electrode 104 at a distal end 106 of the insulated shaft 102. The expandable electrode 104 comprises a shape memory alloy lattice, and based on passing electrical current through the shape memory alloy lattice, the shape memory alloy lattice undergoes a shape memory transition from a non-expanded configuration to an expanded configuration causing the expandable electrode 104 to radially expand outward in diameter.

In FIG. 1, the expandable electrode 104 is shown in the non-expanded configuration. In the non-expanded configuration, the expandable electrode 104 has a circumference or outer diameter the same as or substantially the same as the insulated shaft 102 (e.g., to have the same dimensions) to easily fit into an introducer.

The insulated shaft 102 comprises a conductor with insulation 108 enabling conduction of the electrical current from a proximal end 110 of the insulated shaft 102 to the expandable electrode 104 at the distal end 106 of the insulated shaft 102. In some examples, the insulated shaft 102 and the expandable electrode 104 are formed on the same conductor. The insulated shaft 102 and the expandable electrode 104 are thus one integral component, or are formed on a single piece of material (with the insulated shaft 102 having the insulation 108 later applied).

FIG. 2 illustrates an example of the surgical instrument 100 including the insulated shaft 102 without the insulation 108 and the expandable electrode 104 formed on the same conductor as a single integral component, according to an example implementation. The surgical instrument 100 is a hollow conductor, in this example. The expandable electrode 104 has a flexible tip that is not fully rigid and is an open-ended tip, for example. This enables some flexibility of the tip during operation.

FIG. 3 illustrates an example of a shape memory alloy lattice 112 of the expandable electrode 104, according to an example implementation. The shape memory alloy lattice 112 comprises a plurality of geometric unit cells repeated in circumferential and axial directions along a longitudinal axis of the expandable electrode 104, as shown in FIG. 4. An example unit cell 114 includes a serpentine structure coupled to a circular structure. The serpentine structure and the circular structure are positioned horizontally adjacent each other, and a pattern of the unit cell 114 is repeated vertically such that the circular structure of adjacent unit cells are arranged in the same column and the serpentine structure of adjacent unit cells are arranged in the same column. The serpentine structure includes, in some examples, a compressed “S” shape, or a formation of curves back and forth with an end coupling to the circular structure. In other examples, the circular structure is positioned between two columns of serpentine structures, and is coupled or connected to two adjacent serpentine structures positioned horizontal to the circular structure.

Within examples, the geometric unit cells are arranged in rows and columns in the circumferential manner shown in FIG. 2. Spacing between rows and columns, and within the serpentine structure and the circular structure enables expansion of the shape memory alloy lattice 112. In the non-expanded configuration, shown in FIG. 3, the shape memory alloy lattice 112 is in a compact configuration with each unit cell adjacent one another. The shape memory alloy lattice 112 is laser cut from the conductor material, such that the expandable electrode 104 is formed at the distal end 106 of the insulated shaft 102.

In some examples, the shape memory alloy lattice 112 is formed from a Nitinol alloy material. Nitinol is an alloy of nickel and titanium, and includes shape memory features such that when heated, the Nitinol expands to a predetermined shape. Other shape memory alloys, which are deformed when cold or temperature reduced and return to pre-deformed/pre-set original (“remembered”) shape when heated or temperature increased, are possible as well. Another example shape memory alloy includes copper-aluminium-nickel.

The shape memory alloy lattice 112 is thus a pre-formed Nitinol lattice. The Nitinol lattice shape is pre-formed into the expanded configuration by setting the shape memory alloy lattice into the expanded configuration and then heating the expanded shape memory alloy lattice to about 500 degrees C. (about indicating more or fewer than 50-100 degrees C.) for about 5 minutes (about indicating more or fewer than 2-3 minutes) to set the shape. Then, the preferred shape is set and can be activated when the Nitinol lattice is subsequently heated above a threshold temperature, such as about 70 degrees C. (about indicating more or fewer than 5-10 degrees C. such that higher than human body temperature and heat of patient does not cause expansion, but rather, heat from radio waves or applied electrical current cause expansion).

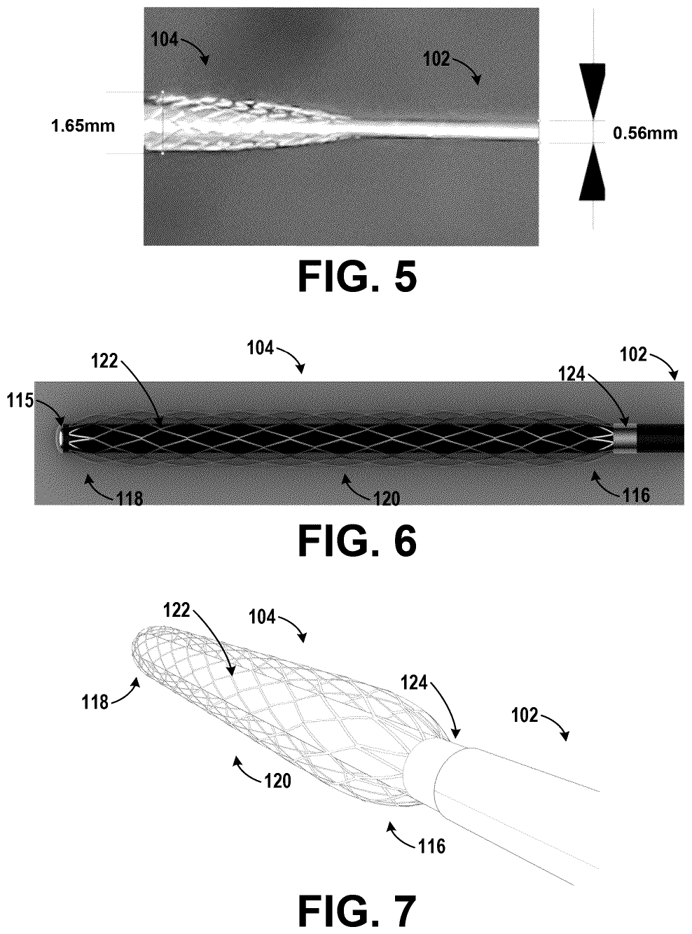

FIG. 5 illustrates an example of the expandable electrode 104 in an expanded configuration, according to an example implementation. The expandable electrode 104 has a length of about 100 mm (e.g., 80-120 mm). In the non-expanded configuration, the expandable electrode 104 has an outer diameter of about 0.56 mm (0.4-0.7 mm), which matches an outer diameter of the insulated shaft 102. In the expanded configuration, the expandable electrode 104 has an outer diameter of about 1.65 mm (1.2-2.1 mm), or about three times as long as the outer diameter when in the non-expanded configuration.

Thus, in examples, the non-expanded configuration of the expandable electrode 104 has an outer diameter of about a 22 gauge needle (e.g., 20-24 gauge), and the expanded configuration of the expandable electrode 104 has an outer diameter of about a 16 gauge needle (e.g., 14-18 gauge).

The expandable electrode 104 may be in many different configurations as well. FIG. 6 illustrates another example of the expandable electrode 104 in an expanded configuration, according to an example implementation. In FIG. 6, the expandable electrode 104 has a plurality of geometric unit cells repeated in circumferential and axial directions along a longitudinal axis of the expandable electrode 104, and the unit cells form a different shape and structure than as shown in FIGS. 1-5. In FIG. 6, the unit cells form a repeating lattice structure similar to a netting configuration that can expand and contract. The expandable electrode 104 in FIG. 6 has an end cap 115. The expanded configuration of the expandable electrode 104 in FIG. 6 includes a first tapered section 116 coupled to the insulated shaft 102, a second tapered section 118 at a distal tip adjacent the end cap 115, and a substantially cylindrical section 120 between the first tapered section 116 and the second tapered section 118.

The expandable electrode 104 in FIG. 6 further includes a central rod 122 for support that couples to the shaft 102. In the example of the expandable electrode 104 in FIG. 6, the expandable electrode 104 is a component comprising the Nitonol alloy lattice that is separate from the shaft 102. A support ring 124 connects the expandable electrode 104 to the shaft 102

FIG. 7 illustrates another example of the expandable electrode 104 shown in FIG. 6, according to an example implementation.

Within examples herein, electrical current is applied to the expandable electrode 104 to cause the shape change. For example, based on passing the electrical current through the shape memory alloy lattice, a temperature of the shape memory alloy lattice increases causing the shape memory transition. Subsequently, based on removal of the electrical current from the shape memory alloy lattice, the shape memory alloy lattice returns to the non-expanded configuration.

With regard to Nitinol and the mechanism of expansion, when electrical current passes through the pre-formed Nitinol, the electrical current causes the material to undergo a phenomenon known as the shape memory effect. Nitinol is a unique alloy made from a combination of nickel and titanium, and exhibits an ability to remember an original pre-formed shape and return to the original pre-formed shape when subjected to certain stimuli, such as heat or electrical current.

In the case of electrical current, the process is as follows: Nitinol is in a martensitic phase at lower temperatures, where Nitinol has a deformed or “memoryless” structure. However, when an electrical current is applied, the electrical current generates heat in the Nitinol wire. The heat causes the material to undergo a phase transformation, transitioning from the martensitic phase to the austenitic phase.

As Nitinol transitions to the austenitic phase, the Nitinol material experiences a crystallographic change, allowing the Nitinol material to regain an original pre-formed shape. The shape recovery occurs due to a rearrangement of an atomic structure of the material. Expansion of the Nitinol wire is a result of this shape memory effect.

Expansion of Nitinol due to application of electrical current is only temporary and reversible. Once the electrical current is removed, and the Nitinol cools down in temperature, the Nitinol will return to its deformed shape.

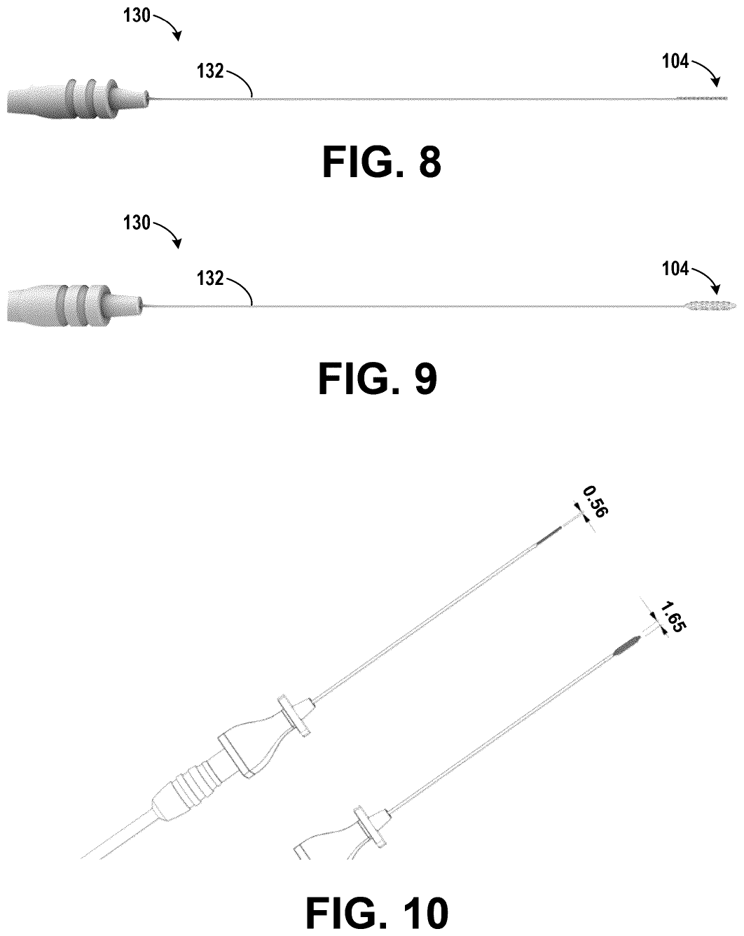

FIG. 8 illustrates an example of surgical equipment 130, according to an example implementation. The surgical equipment 130 comprises an introducer 132, and the surgical instrument 100 inserted into the introducer 132. The surgical instrument 100 takes the form as described with respect to any of FIGS. 1-7, and comprises the insulated shaft, and the expandable electrode 104 at a distal end of the insulated shaft. The expandable electrode comprises a shape memory alloy lattice, and based on passing electrical current through the shape memory alloy lattice, the shape memory alloy lattice undergoes a shape memory transition from a non-expanded configuration to an expanded configuration causing the expandable electrode to radially expand outward in diameter.

In FIG. 8, the expandable electrode 104 is shown in non-expanded configuration (compressed form). FIG. 9 illustrates the surgical equipment 130 of FIG. 8 with the expandable electrode 104 in the expanded configuration. With passage of electrical energy to and along the expandable electrode 104, the Nitinol tip expands to a pre-formed cylindrical shape. When the electrical current is discontinued, the Nitinol tip reverts to the compressed state and can be withdrawn through the introducer 132.

FIG. 10 illustrates comparative dimensions (in mm) of the expandable electrode 104, according to an example implementation. The compressed tip in the non-expanded configuration has an outer diameter of about 0.56 mm that approximates to a 22G needle diameter, and the expanded configuration has an outer diameter of about 1.65 mm that approximates to a 16G needle diameter, for example.

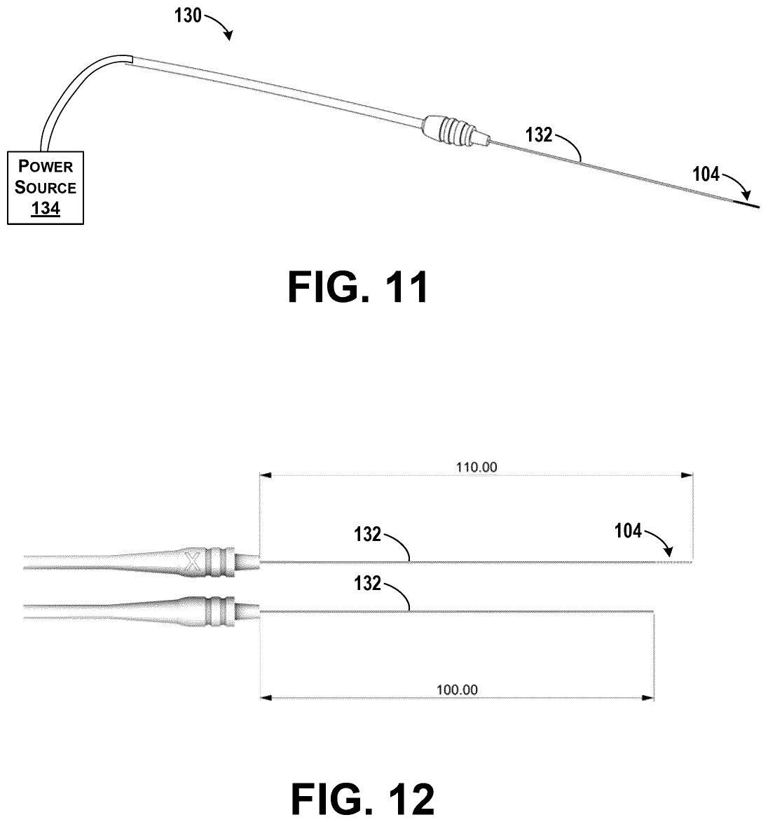

FIG. 11 illustrates another example of the surgical equipment 130, according to an example implementation. The surgical equipment 130 further includes a power source 134 coupled to the surgical instrument 100 to provide the electrical current to the expandable electrode 104.

FIG. 12 illustrates another example of the surgical equipment with the expandable electrode 104 in an extended and retracted position. The surgical instrument 100 is selectively translatable relative to the introducer 132 between a proximal retracted position in which the expandable electrode is housed within the introducer 132 (shown on bottom view of FIG. 12) to a distal extended position in which the expandable electrode 104 is exposed from the introducer 132 (shown on top view of FIG. 12) to permit the expandable electrode 104 to radially expand from the non-expanded configuration to the expanded configuration. In the extended position, the expandable electrode 104 extends out of the distal end of the introducer by about 10 mm, for example.

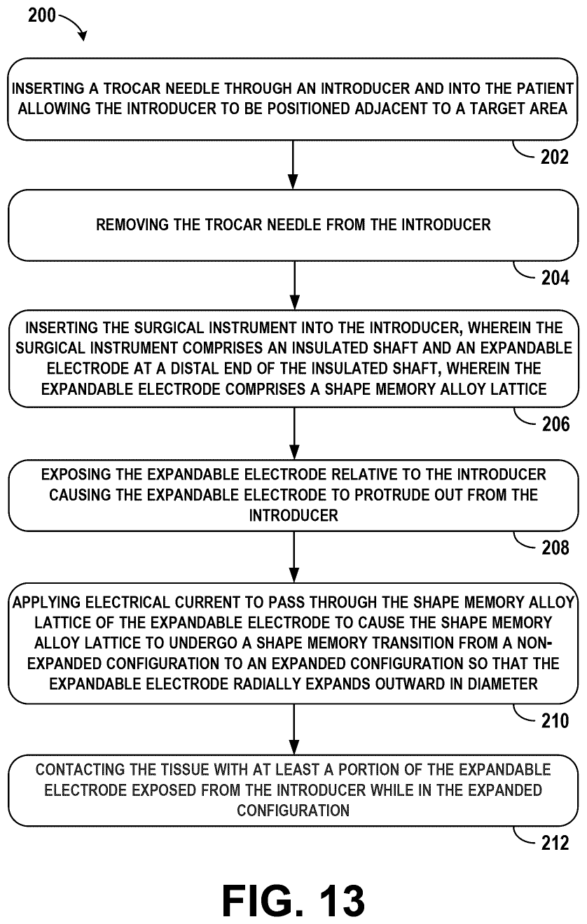

FIG. 13 shows a flowchart of an example of a method 200 of ablating tissue in a patient with a surgical instrument, according to an example embodiment. The method shown in FIG. 13 presents an example of a method that, for example, could be implemented by the surgical instrument 100 shown in FIGS. 1-7 and the surgical equipment 130 shown in FIGS. 8-12, for example. In some instances, components of the surgical instrument 100 are configured to perform the functions such that the components are actually configured and structured (with hardware and/or software) to enable such performance. In other examples, components of the devices and/or systems are arranged to be adapted to, capable of, or suited for performing the functions, such as when operated in a specific manner. The method may include one or more operations, functions, or actions as illustrated by one or more of blocks 202-212. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In still further examples, some of the functions of the method 200 described herein are performed by circuitry (e.g., a processor) executing instructions stored on non-transitory computer-readable medium to cause the surgical instrument 100 to provide stimulation treatment.

It should be understood that for this and other processes and methods disclosed herein, flowcharts show functionality and operation of one possible implementation of present embodiments. Alternative implementations are included within the scope of the example embodiments of the present disclosure in which functions may be executed out of order from that shown or discussed, including substantially concurrent or in reverse order, depending on the functionality involved, as would be understood by those reasonably skilled in the art.

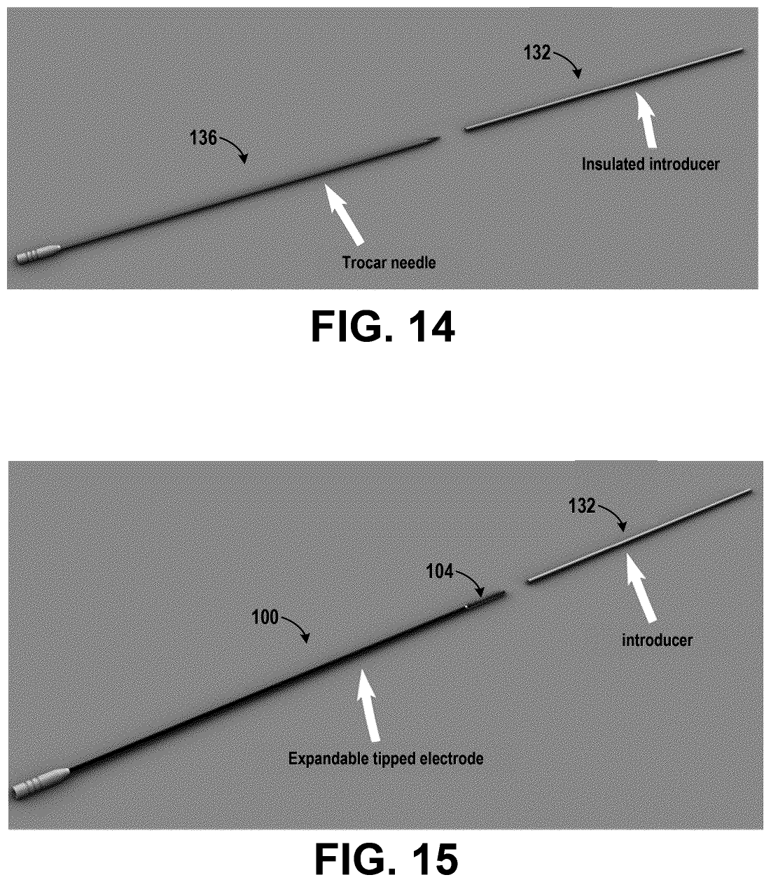

At block 202, the method 200 includes inserting a trocar needle through an introducer and into the patient allowing the introducer to be positioned adjacent to a target area. FIG. 14 illustrates an example of inserting a trocar needle 136 through the introducer 132, which is then subsequently inserted into a patient allowing the introducer 132 to be positioned adjacent to a target area, according to an example implementation.

Referring back to FIG. 13, at block 204, the method 200 includes removing the trocar needle 136 from the introducer 132.

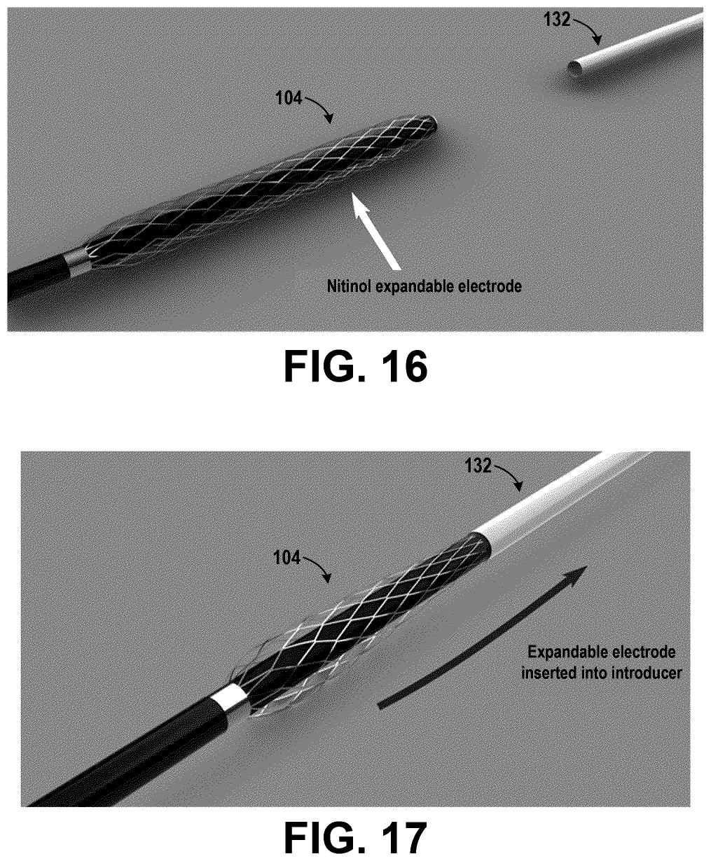

At block 206, the method 200 includes inserting the surgical instrument 100 into the introducer 132, and the surgical instrument 100 comprises the insulated shaft 102 and the expandable electrode 104 at the distal end 106 of the insulated shaft 102. The expandable electrode 104 comprises a shape memory alloy lattice. FIG. 15 illustrates an example of inserting the surgical instrument 100 into the introducer 132, according to an example implementation. FIG. 16 illustrates another example of inserting the expandable electrode 104 into the introducer 132, and FIG. 17 illustrates yet another view of inserting the surgical instrument 100 into the introducer 132, according to an example implementation.

Referring back to FIG. 13, at block 208, the method 200 includes exposing the expandable electrode 104 relative to the introducer 132 causing the expandable electrode 104 to protrude out from the introducer 132.

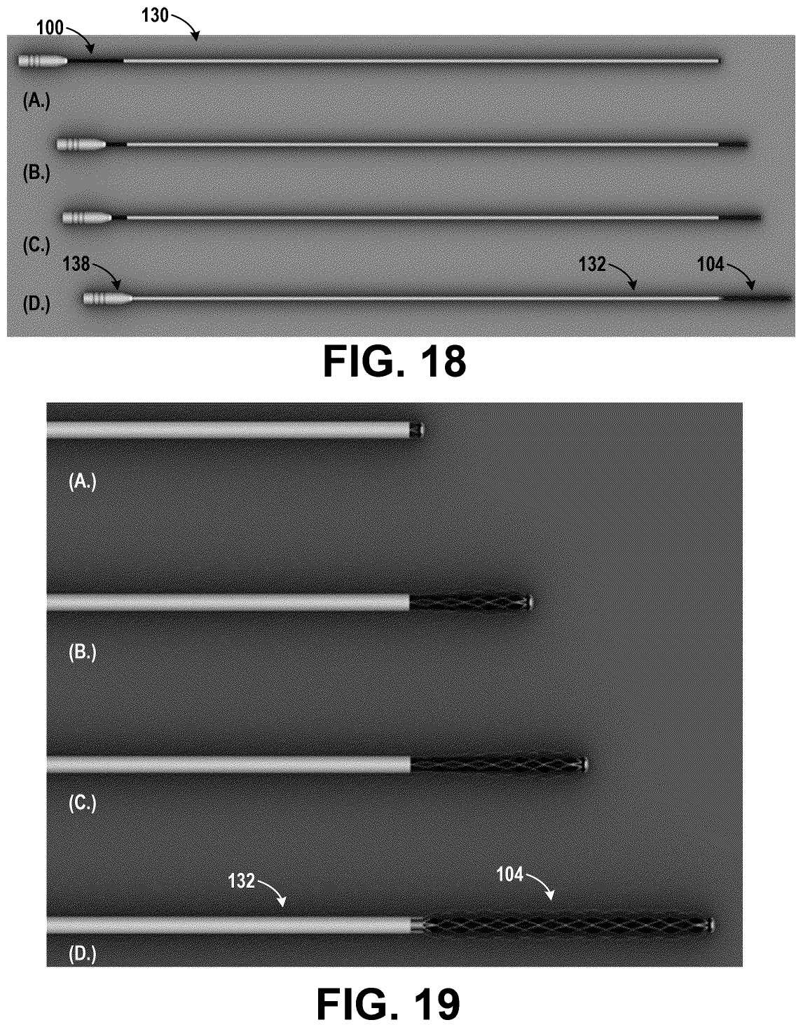

FIG. 18 illustrates an example of stages of exposing the expandable electrode 104 relative to the introducer 132, according to an example implementation. FIG. 19 illustrates yet another view of stages of exposing the expandable electrode 104 relative to the introducer 132, according to an example implementation. With reference to FIGS. 18-19, the expandable electrode 104 is exposed relative to the introducer 132 by pushing the surgical instrument 100 farther into the introducer 132, as shown in steps (A.) through (D.). The expandable electrode 104 begins to protrude out of an end of the introducer 132 and continues to do so to the extent of a length of the surgical instrument 100 relative to a length of the introducer 132. Once a handle 138 of the surgical instrument 100 abuts a proximal end of the introducer 132, as shown in step (D.), the expandable electrode 104 is exposed to a full extent, which may be about 10 mm, for example.

Referring back to FIG. 13, at block 210, the method 200 includes applying electrical current to pass through the shape memory alloy lattice of the expandable electrode 104 to cause the shape memory alloy lattice to undergo a shape memory transition from a non-expanded configuration to an expanded configuration so that the expandable electrode radially expands outward in diameter.

At block 212, the method 200 includes contacting the tissue with at least a portion of the expandable electrode exposed from the introducer while in the expanded configuration. Following, RF energy is supplied to the expandable electrode 104 to cause ablation of tissue.

Within examples, after the expandable electrode 104 receives the electrical current and generates heat, based on contacting the tissue with the portion of the expandable electrode 104 exposed from the introducer 132 while in the expanded configuration results in forming a lesion circumferentially along a body of the expandable electrode.

In some examples, the method 200 additionally includes detecting a temperature of the expandable electrode 104 with a thermistor, and based on the temperature being above a threshold, discontinuing application of the electrical current to the shape memory alloy lattice. For instance, a thermistor is positioned at a tip of the expandable electrode 104 to detect the temperature.

In some examples, the method 200 additionally includes after said contacting, discontinuing application of the electrical current to the shape memory alloy lattice to cause the shape memory alloy lattice to transition from the expanded configuration to the non-expanded configuration.

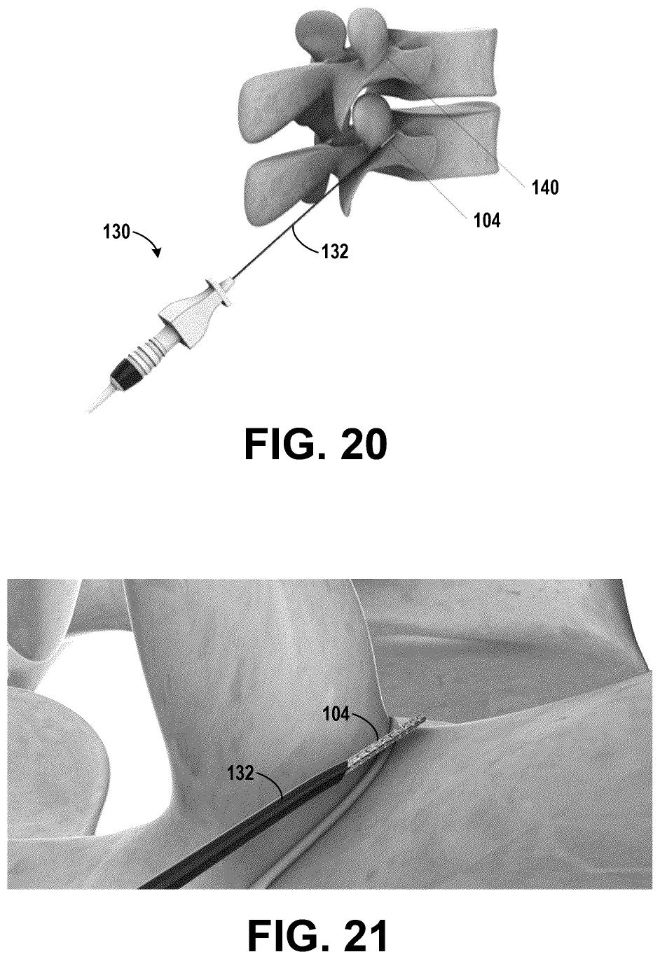

FIGS. 20-23 illustrate application of the surgical equipment 130 for treatment of pain for RF ablation of a lumbar facet joint, according to an example implementation. In FIGS. 20-21, the surgical instrument 100 is inserted into the introducer 132 and the expandable electrode 104 is exposed and in the non-expanded configuration. FIG. 20 illustrates the deployed expandable system with the compressed tip positioned parallel to a medial branch nerve 140 supplying a zygo-apophyseal spinal joint. FIG. 21 illustrates a magnified view of the exposed metallic contact of the expandable electrode 104 that will allow passage of RF current to the nerve.

An insulated distal portion of the expandable electrode 104 coupled to the insulated shaft maintains rigidity without generation of RF current, while the expandable electrode 104 in the non-expanded configuration includes the elongated Nitinol lattice. Thus, FIG. 21 illustrates a detailed view of the expandable electrode 104 in a compressed form protruding distally from the introducer 132 and lying adjacent to the medial branch nerve 140.

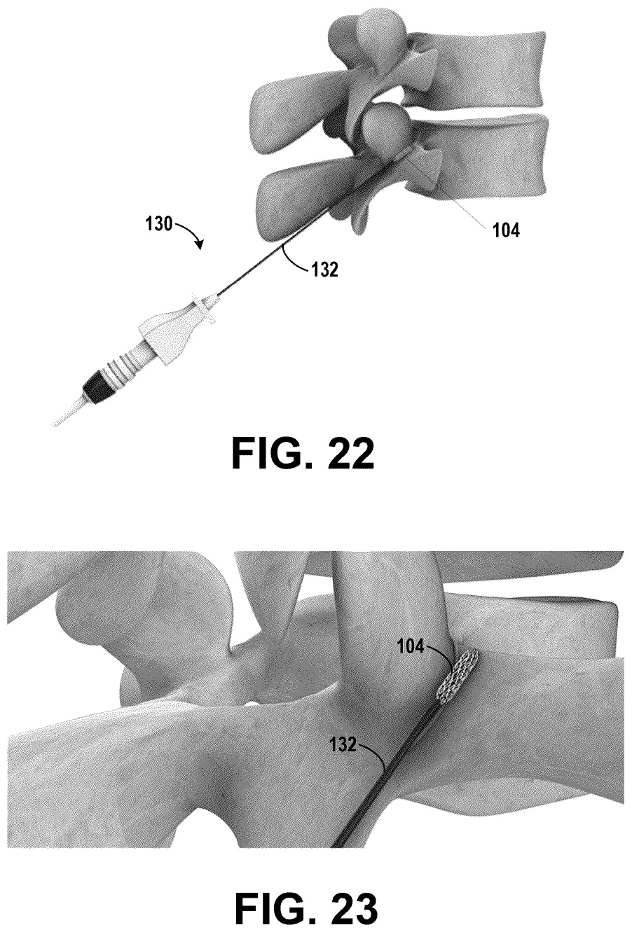

In FIGS. 22-23, the expandable electrode 104 is in the expanded configuration lying parallel to the medial branch nerve 140. FIG. 22 illustrates the deployed expandable system with the expanded tip positioned parallel to the medial branch nerve 140, and FIG. 23 illustrates a magnified view of the expandable electrode 104 in expanded form (following passage of electrical energy along the expandable electrode 104) lying parallel to the medial branch nerve 140.

By positioning the introducer 132 next to the nerve 140 and inserting the surgical instrument 100 into the introducer 132, the expandable electrode 104 can be positioned adjacent to the target nerve. Following, electrical current is passed to the expandable electrode 104 to cause the expandable electrode 104 to transition to the expanded configuration (as shown in FIG. 23) so that the expandable portion protrudes from the tip of the introducer 132 and the exposed electrode expands to a pre-set diameter. Subsequently, radio wave current is passed down to the expanded electrode to generate heat at the tip, such as heated to a temperature of about 80 to 90 degrees C. Following, the RF current is discontinued, and the surgical instrument 100 is retracted into the introducer 132 for removal.

The procedure is performed under local anesthesia, and thus, it is desirable to use as small a needle as possible. However, smaller needles can only be used to create smaller ablations or lesions. Thus, usage of the example surgical instrument 100 described herein allow for insertion of a small diameter needle and creation of a lesion using radiofrequency substantially equivalent to a larger diameter needle size.

Example implementations of the surgical instrument 100 described herein are single use devices or medical instruments. Radio frequency energy, when applied, generates heat at the tip of the expandable electrode so that all heat is contained within an area of the tip and the electrode is disposed of after use.

Example indications and intended uses of the surgical instrument 100 include pain treatment (both chronic and acute), inflammation, and neuromodulation of nerve functions.

By the term “about” and/or the term “substantially” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide. Example tolerances include 5% differences or more or less than the stated amounts.

It should be emphasized that the above-described embodiments of the present disclosure are merely possible examples of implementations set forth for a clear understanding. Many variations and modifications may be made to the above-described embodiment(s) without departing substantially from the principles of the disclosure. All such modifications and variations are intended to be included herein within the scope of this disclosure.

What is claimed is:

1. A surgical instrument, comprising:
an insulated shaft; and
an expandable electrode at a distal end of the insulated shaft, wherein the expandable electrode comprises a shape memory alloy lattice, and based on passing electrical current through the shape memory alloy lattice, the shape memory alloy lattice undergoes a shape memory transition from a non-expanded configuration to an expanded configuration causing the expandable electrode to radially expand outward in diameter.

2. The surgical instrument of claim 1, wherein the shape memory alloy lattice is a pre-formed nitinol lattice.

3. The surgical instrument of claim 1, wherein based on removal of the electrical current from the shape memory alloy lattice, the shape memory alloy lattice returns to the non-expanded configuration.

4. The surgical instrument of claim 1, wherein based on passing the electrical current through the shape memory alloy lattice, a temperature of the shape memory alloy lattice increases causing the shape memory transition.

5. The surgical instrument of claim 1, wherein the expandable electrode has a length of about 100 mm.

6. The surgical instrument of claim 1, wherein the expanded configuration of the expandable electrode has an outer diameter of about 1.65 mm.

7. The surgical instrument of claim 1, wherein the non-expanded configuration of the expandable electrode has an outer diameter of about a 22 gauge needle.

8. The surgical instrument of claim 1, wherein the expanded configuration of the expandable electrode has an outer diameter of about a 16 gauge needle.

9. The surgical instrument of claim 1, wherein the expanded configuration of the expandable electrode includes:
a first tapered section coupled to the insulated shaft;
a second tapered section at a distal tip; and
a substantially cylindrical section between the first tapered section and the second tapered section.

10. The surgical instrument of claim 1, wherein the expandable electrode has a flexible tip.

11. The surgical instrument of claim 1, wherein the insulated shaft comprises a conductor with insulation enabling conduction of the electrical current from a proximal end of the insulated shaft to the expandable electrode at the distal end of the insulated shaft.

12. The surgical instrument of claim 1, wherein the insulated shaft comprises a conductor and the expandable electrode is formed at the distal end of the conductor, such that the insulated shaft and the expandable electrode are formed on the same conductor.

13. The surgical instrument of claim 1, wherein the shape memory alloy lattice comprises:
a plurality of geometric unit cells repeated in circumferential and axial directions.

14. A surgical equipment, comprising:
an introducer;
a surgical instrument inserted into the introducer, wherein the surgical instrument comprises:
an insulated shaft; and
an expandable electrode at a distal end of the insulated shaft, wherein the expandable electrode comprises a shape memory alloy lattice, and based on passing electrical current through the shape memory alloy lattice, the shape memory alloy lattice undergoes a shape memory transition from a non-expanded configuration to an expanded configuration causing the expandable electrode to radially expand outward in diameter.

15. The surgical equipment of claim 14, wherein:
the surgical instrument is selectively translatable relative to the introducer between a proximal retracted position in which the expandable electrode is housed within the introducer to a distal extended position in which the expandable electrode is exposed from the introducer to permit the expandable electrode to radially expand from the non-expanded configuration to the expanded configuration.

16. The surgical equipment of claim 14, further comprising:
a power source coupled to the surgical instrument to provide the electrical current to the expandable electrode.

17. A method of ablating tissue in a patient with a surgical instrument, comprising:
inserting a trocar needle through an introducer and into the patient allowing the introducer to be positioned adjacent to a target area;
removing the trocar needle from the introducer;
inserting the surgical instrument into the introducer, wherein the surgical instrument comprises an insulated shaft and an expandable electrode at a distal end of the insulated shaft, wherein the expandable electrode comprises a shape memory alloy lattice;
exposing the expandable electrode relative to the introducer causing the expandable electrode to protrude out from the introducer;
applying electrical current to pass through the shape memory alloy lattice of the expandable electrode to cause the shape memory alloy lattice to undergo a shape memory transition from a non-expanded configuration to an expanded configuration so that the expandable electrode radially expands outward in diameter; and
contacting the tissue with at least a portion of the expandable electrode exposed from the introducer while in the expanded configuration.

18. The method of claim 17, wherein the expandable electrode receives the electrical current and generates heat, and wherein based on contacting the tissue with the portion of the expandable electrode exposed from the introducer while in the expanded configuration results in forming a lesion circumferentially along a body of the expandable electrode.

19. The method of claim 17, further comprising:
detecting a temperature of the expandable electrode with a thermistor; and
based on the temperature being above a threshold, discontinuing application of the electrical current to the shape memory alloy lattice.

20. The method of claim 17, further comprising:
after said contacting, discontinuing application of the electrical current to the shape memory alloy lattice to cause the shape memory alloy lattice to transition from the expanded configuration to the non-expanded configuration.
